(19) United States
(12) Patent Application Publication
(10) Pub. No.: US 2007/0066814 A1
(43) Pub. Date: Mar. 22, 2007

(54) Steroid modified chacotrioses and solatrioses

(75) Inventors: Christopher John Lawson, Reading (GB); Alexander Charles Weymouth-Wilson, Reading (GB)

Correspondence Address: KAGAN BINDER, PLLC, SUITE 200, MAPLE ISLAND BUILDING, 221 MAIN STREET NORTH, STILLWATER, MN, 55082, US

(73) Assignee: GLYCOMED SCIENCES LIMITED, Turramurra NSW (KY)

(21) Appl. No.: 10/563,743

(22) PCT Filed: Jul. 8, 2004
(86) PCT No.: PCT/EP04/07537

§ 371 Date: Jul. 10, 2006

(30) Foreign Application Priority Data
Jul. 8, 2003 (EP) 0301550202

(51) Int. Cl. C07J 17/00

(52) U.S. Cl. 536/6.1

(57) ABSTRACT

The present invention relates to steroid modified chacotrioses and the synthesis thereof as well as to intermediate compounds useful for the synthesis of the steroid modified chacotrioses and solatrioses. Moreover, the present inventions relates to a method for the preparation of steroid-modified solatrioses.

The present invention relates to the chemical synthesis of alkaloid glycosides, in particular to the synthesis of steroid modified chacotrioses and solatrioses. Furthermore, the present invention relates to intermediate compounds useful for the preparation of steroid modified chacotrioses and solatrioses and to novel steroid modified chacotrioses.

The aglycon solasodine is a source for synthetic cortisone and progesterone. Solasodine and its glycosides are of considerable interest commercially and clinically. They are widely used as starting products for the synthesis of various steroidal drugs.

It is moreover well established that certain naturally occurring conjugate solasodine glycosides have potent antineoplastic properties. Of particular interest is the chacotriose type triglycoside solamargine (22R, 25R)-spiro-5-en-3β-yl-α-L-rhamnopyranosyl-(1->2 glu)-α-L-rhamnopyranosyl- (1->4 glu)-β-D-gluco-pyranose. The structure of this triglycoside is as follows:

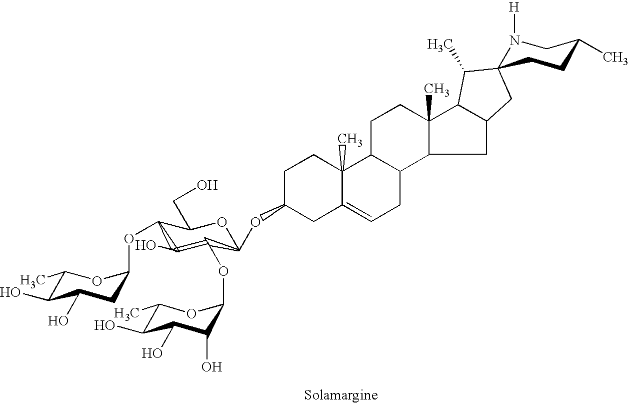

Another naturally occurring conjugate solasodine glycoside of particular interest is the solatriose type triglycoside solasonine (22R, 25R)-spiro-5-en-3β-yl-α-L-rhamno-pyranosyl-(1->2 gal)-O-p-D-glucopyranosyl-(1->3 gal)-β-D-galactopyranose. The structure of this triglycoside is as follows:

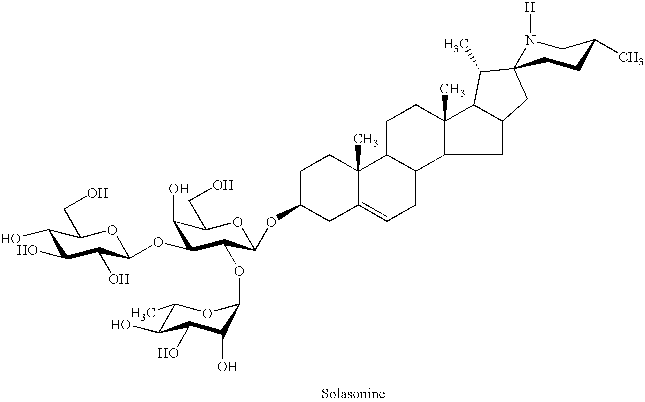

The above triglycosides are conventionally obtained by extraction from a plant source. A commercially available extract of S. sodomaeum, commonly referred to as BEC (Drug Future, 1988, vol. 13.8, pages 714-716) is a crude mixture of solamargine, solasonine and their isomeric diglycosides. The extraction process for making BEC involves homogenizing the fruits of S. sodomaeum in a large volume of acetic acid, filtering off the liquid through muslin followed by precipitation of the glycosides with ammonia (Drugs of today (1990), Vol. 26 No. 1, p. 55-58, cancer letters (1991), Vol. 59, p. 183-192). The yield of the solasodine glycoside mixture is very low (approx. 1%). Moreover the individual process steps are not defined to GMP in terms of scale up, definition of yield, composition and product quality.

There is a great need for a cost efficient process that provides the antineoplastically active triglycosides such as solamargine and solasonine as well as analogues thereof at high yield with little or no impurities.

Contrary to other steroid ring systems, the steroid skeleton of solasodine contains a very labile nitrogen-containing ring. The same hold true for the steroid ring systems of other alkaloids, notably tomatidine, demissidine or solanidine. These aglycons cannot readily be chemically modified while keeping the steroid skeleton intact. In spite of the fact that the aglycon solasodine is readily available, the prior art does not disclose the synthesis of the solamargine or solasonine using the aglycon as starting material.

The problem underlying the present invention is to provide a cost effective method for the preparation of steroid modified chacotrioses and solatrioses such as solamargine and solasonine or analogues thereof in high yields.

Such compounds exhibit cytotoxic activity and may be employed as anticancer agents. Furthermore, such compounds exhibit anti bacterial, anti fungal or anti viral activity.

Accordingly, the present invention provides a method for the preparation of a steroid modified chacotriose of general formula (Ia) or a steroid modified solatriose of general formula (Ib):

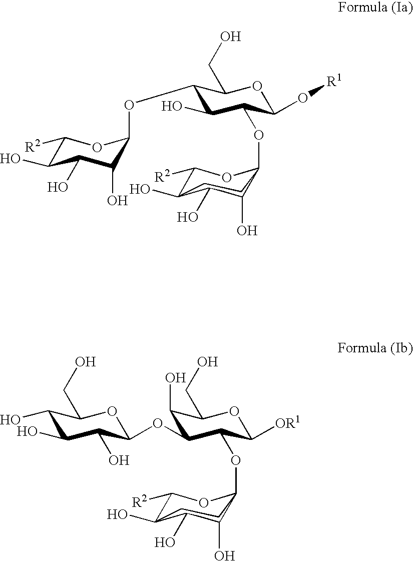

wherein R¹ represents a steroid or a derivative thereof having a hydroxyl group in the 3-position and no further unprotected hydroxyl groups; and each R² independently represents a straight or branched C₁₋₁₄ alkyl group, a C₅₋₁₂ aryl or heteroaryl group optionally substituted by one or more halogen atoms or C₁₋₄ alkyl groups, or a hydroxyl group.

The method comprises the step of: reacting a compound of general formula (IIa) or (IIb):

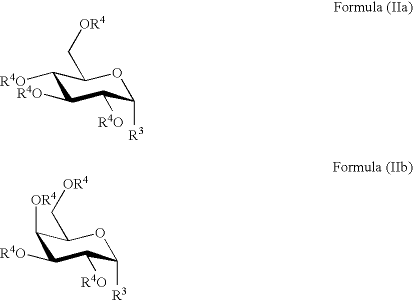

wherein R³ represents a halogen atom, an ethylsulfide or a phenyl sulfide group; and each R⁴ independently represents a benzoyl, substituted benzoyl, whereby the substituents are selected from C₁₋₄ alkyl groups, halogen atoms and NO₂, acetyl or pivolyl protecting group; with a compound of general formula (III):
HO—R¹ Formula (III)

wherein R¹ is defined as above; to yield a compound of general formula (IVa) or (IVb):

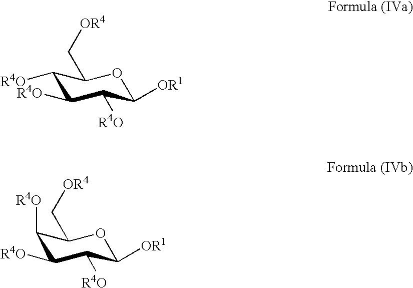

wherein R¹ and R⁴ are defined as above.

The compounds of the above general formulae (IVa) and (IVb) may be transformed to the desired steroid-modified chacotriose of general formula (Ia) or the steroid-modified solatriose of general formula (Ib) by any suitable method known in the art. A particular preferred procedure is described in detail below.

Furthermore, the present application provides steroid modified chacotriose compounds of general formula (Ia) as defined above, wherein R¹ represents a tomatidin-3-yl, demissidin-3-yl group, solanidin-3-yl or solasodin-3-yl group.

A further object of the present application is the provision of intermediate compounds useful for the synthesis of the steroid modified chacotriose of general formula (Ia) defined above, namely:

A compound of general formula (IVa) or (IVb) as defined above;

A compound of general formula (Va) or (Vb):

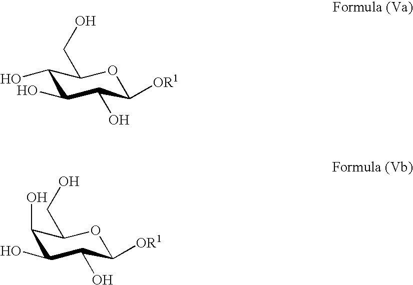

wherein R¹ is defined as above;

A compound of general formula (VIa) or (VIb):

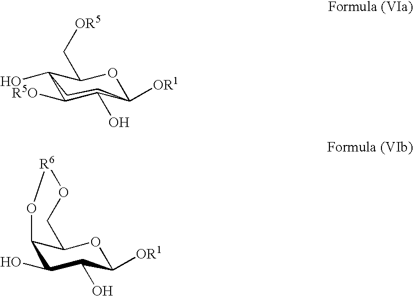

wherein R¹ is as defined above, R⁵ represents a pivolyl protecting group, and R⁶ represents a ketal or acetal type protecting group selected from benzylidene, 4-nitrobenzylidene, 4-methoxybenzylidene or isopropylidene.

A compound of general formula (VIIIa) or (VIIIb):

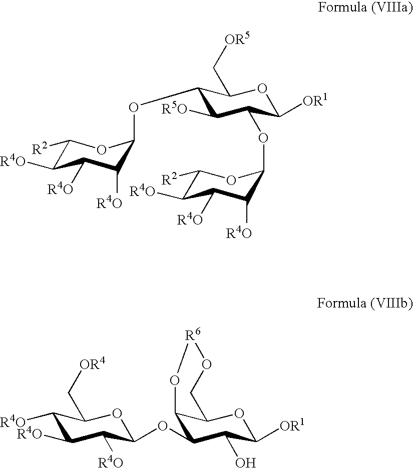

wherein R¹, R², R⁴, R⁵ and R⁶ are as defined above.

A compound of general formula (IXb):

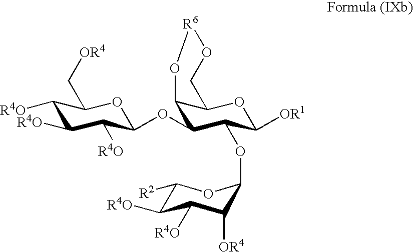

wherein R¹, R², R⁴ and R⁶ are as defined above.

Further embodiments of the present application are described in the dependent claims.

### DETAILED DESCRIPTION OF THE INVENTION

In the following, the present invention will be explained in more detail with reference to preferred embodiments.

The steroid residue constituting substituent R¹ is a steroid or a derivative thereof having a hydroxyl group in the 3-position that serves as the α-glycosidic hydroxyl group, which binds the steroid residue to the compound of formula (II) defined above. The steroid residue bears no further unprotected hydroxyl groups and preferably has no further hydroxyl groups at all, in order not to compromise subsequent reaction steps. In a preferred embodiment of the present invention R¹ is selected from a tomatidin-3-yl, demissidin-3-yl, solanidin-3-yl and solasodin-3-yl group.

All of those steroid groups contain a labile nitrogen-containing ring and, therefore, cannot be chemically modified by means of conventional methods. Moreover, all of the above steroid groups represent substituents for cyctotoxic, anti bacterial, anti fungal or anti viral compounds.

In the above general formulae (Ia) and (Ib) each R² independently represents a straight or branched C₁₋₁₄ alkyl group, a C₅₋₁₂ aryl or heteroaryl group optionally substituted by one or more halogen atoms or C₁₋₄ alkyl groups, or a hydroxyl group. In a preferred embodiment R² represents a C₁₋₁₄ alkyl group selected from methyl, ethyl and propyl; an aryl group selected from phenyl, p-methylphenyl and p-chlorophenyl; or an heteroaryl group selected from pyridinyl, pyrimidinyl, furanyl, pyrrolyl, thiophenyl, indolyl, pyrazolyl and imidazolylmethyl; methyl, ethyl and propyl are more preferred.

In a particular preferred embodiment R² represents a methyl group.

The method of the present invention for preparing a steroid-modified chacotriose of general formula (Ia) comprises reacting a compound of general formula (IIa):

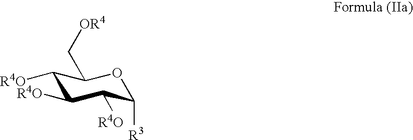

with a compound of general formula (III):
HO—R¹ Formula (Ill)

to yield a compound of general formula (IVa):

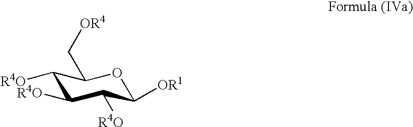

In the above general formula (IIa) R³ represents a halogen atom, an ethylsulfide or a phenyl sulfide group. Preferably, R³ represents a bromine atom or a chlorine atom. Most preferably R³ is a bromine atom. Furthermore, in general formulae (IIa) and (IVa), each R⁴ independently represents a benzoyl, substituted benzoyl, whereby the substituents are selected from C₁₋₄ alkyl groups, halogen atoms and NO₂, acetyl or pivolyl protecting group, preferably a benzoyl or p-toluoyl protecting group, most preferably a benzoyl protecting group.

The above step is preferably conducted in an inert organic solvent such dichloromethane, tetrahydrofuran or dichloroethane. A preferred solvent is dichloromethane.

Preferably the reaction is carried out in the presence of a promoter. Any conventional promoter used in carbohydrate chemistry may be employed. Particular preferred promoters include silver triflate, boron trifluoride diethyl etherate (−10° C.), trimethylsilyl triflate bromide, N-iodosuccinimide and dimethyl thiomethyl sulfonium triflate. The most preferred promoter is silver triflate.

The reaction may preferably be carried out under anhydrous conditions in the presence of a water detracting means such as 4 Å mol sieves.

In a preferred embodiment the reaction is carried out at low temperature such as 0° C. or lower, more preferably −10° C. or lower. The most preferred reaction temperature is −20° C.

Subsequently, the above-obtained compound of general formula (IVa) may be further modified as described below.

In a preferred embodiment of the method of the present application, the compound of general formula (IVa) is deprotected by removing substituent R⁴ to obtain a compound of general formula (Va):

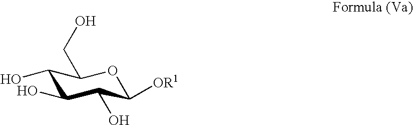

wherein R¹ is defined as above.

Any suitable deprotection condition conventionally employed in the chemistry of protecting groups may be used. Deprotection is preferably carried out in an inert organic solvent such as dichloromethane or tetrahydrofuran in the presence of an alkali metal alkoxide having 1 to 4 carbon atoms and a C₁₋₄ alcohol, or in the presence of water, an alkali metal hydroxide and a C₁₋₄ alcohol. In a particular preferred embodiment deprotection is carried out in dichloromethane in the presence of methanol and sodium methoxide.

The thus obtained compound of general formula (Va) may be selectively protected in 3-OH and 6-OH position using pivolyl chloride in the presence of an amine base to yield compound of general formula (VIa):

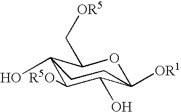

### Formula (VIa)

wherein R¹ is as defined above, and R⁵ represents a pivolyl group. Suitable amine bases include pyridine, triethylamine, collidine, or lutidine. A preferred amine base is pyridine.

The reaction may be carried out in an inert organic solvent. Examples of suitable solvents include tetrahydrofuran, dichloroethane, or dimethylformamide.

The compound of formula (VIa) may be then reacted with a compound of general formula (VIIa):

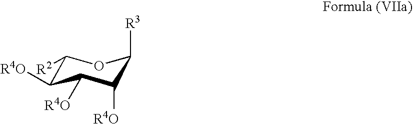

under substantially the same conditions as described above for the preparation of the compound of formula (IVa). In general formula (VIIa) R², R³ and R⁴ are as defined above.

Resulting compound of general formula (VIIIa):

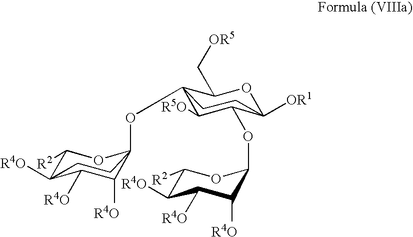

wherein R¹, R², R⁴ and R⁵ are as defined above, may be subsequently deprotected to yield the compound of general formula (la) under substantially the same conditions as described above for the preparation of the compound of formula (Va). In a preferred this deprotection step is carried out in tetrahydrofuran in the presence of water, sodium hydroxide and methanol.

In another embodiment, the present invention provides a method for preparing a steroid-modified solatriose of general formula (Ib). According to a preferred embodiment of the method for preparing a steroid-modified solatriose of general formula (Ib), galactose is reacted to yield a compound of general formula (IIb):

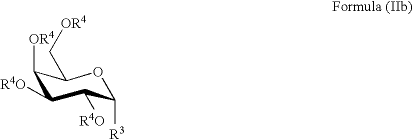

wherein R³ and R⁴ are as defined above.

The preparation of the compound of formula (IIb) may be carried out using either acetic anhydride, acetyl chloride, benzoyl chloride, benzoic anhydride, or pivolyl chloride in the presence of a base such as, e.g., pyridine, triethylamine, or collidine, to give fully esterified galactose. Esterified-D-galactopyranose may be treated with hydrogenbromide or hydrogenchloride in glacial acetic acid to yield the above compound of general formula (IIb).

In a particularly preferred embodiment galactose is suspended in organic base such as pyridine and cooled to 0° C., to this solution is added dropwise either acetic anhydride, benzoic anhydride or acid chloride. Upon complete addition the solution is warmed to +25° C. (room temperature) and stirred for about 16 hours. The reaction is quenched by addition of alcohol. The solution is diluted with organic solvent such as tert-butylmethyl ether, or dichloromethane, or toluene and washed with cold 1N HCl, water, saturated sodium bicarbonate, water and brine then the product is dried over magnesium sulfate and concentrated under reduced pressure to dryness. The product can be used without further purification or it can be recrystallised.

The fully esterified galactopyranose in dry solvent such as dichloromethane is cooled to 0° C. under an inert atmosphere. To this solution is added hydrogen bromide in glacial acetic acid, typically 30% HBr content. The solution is allowed to warm to +25° C. (room temperature) and stirred for around 16 hours. The solution is diluted with organic solvent such as dichloromethane and then quickly washed with ice cold water, saturated aqueous sodium bicarbonate, and brine. The product is dried over magnesium sulfate filtered and the solvent is removed under reduced pressure. The product is crystallized from petrol (40-60) and diethyl ether.

Furthermore, the method for preparing a steroid-modified solatriose of general formula (Ib) comprises reacting the compound of general formula (IIb) as defined above with a compound of general formula (III) as defined above to yield a compound of general formula (IVb):

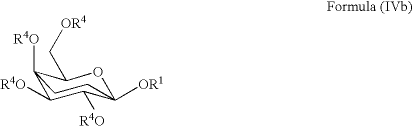

in which R³ and R⁴ are as defined above.

The step for preparing the compound of formula (IVb) is preferably conducted under substantially the same conditions as the reaction for preparing the compound of formula (IVa) above.

Alternatively, the reaction may be carried out by reacting the compound of formula (III) as defined above with intermediate (A):

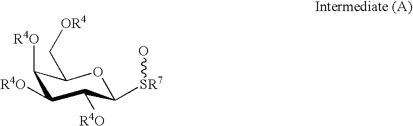

wherein R⁴ is defined above, and R⁷ represents any alkyl or aryl residue, e.g., a straight or branched C₁₋₁₄ alkyl group or a phenyl group optionally substituted with one or more C₁₋₄ alkyl groups; whereby the C₁₋₁₄ alkyl group is preferably selected from methyl, ethyl and propyl and the phenyl group is preferably selected form phenyl, p-methylphenyl and p-chlorophenyl. The reaction can be carried out in a suitable solvent such as dichloromethane or a combination of dichloromethane and an ether such as diethylether. The reaction is preferably carried out in the presence of a promoter as defined above, e.g., triflic anhydride, and a sterically hindered base such as 2,6-lutidine, 2,4,6-collidine or 2,6-di-tertbutyl-4-methyl pyridine, preferably 2,6-di-t-butylpyridine, at low temperature (below −10° C., preferably below −20° C.).

In this embodiment, intermediate (A) may be obtained by oxidizing intermediate (B):

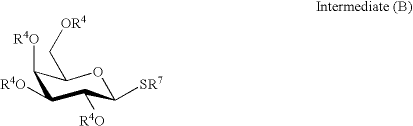

wherein R⁴ and R⁷ are as defined above, to yield the corresponding sulfoxide (i.e., intermediate (A)). Oxidation of intermediate (B) may be effected using a suitable oxidation means, e.g., m-chloroperbenzoic acid. The reaction may be carried out in a solvent such as dichloromethan at low temprature (−20° C., preferably −40° C.).

Intermediate (B) may be formed by the treatment of the compound of formula (IIb) with an alkali metal salt of an alkyl or aryl thiol (R⁷—SH), e.g., the potassium or sodium salt of R⁷—SH, in a suitable solvent such as ethanol or methanol.

Subsequently, the above-obtained compound of general formula (IVb) may deprotected by removing substituent R⁴ to obtain a compound of general formula (Vb):

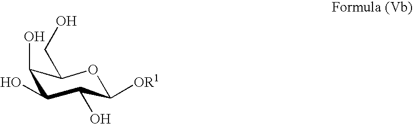

wherein R¹ is defined as above.

Any suitable deprotection condition conventionally employed in the chemistry of protecting groups may be used. In particular, deprotection may preferably be carried out as described above for the preparation of the compound of formula (Va).

The thus obtained compound of general formula (Va) may be selectively protected in 4-OH and 6-OH position with a ketal or acetal protecting group using standard conditions to yield a compound of general formula (VIb):

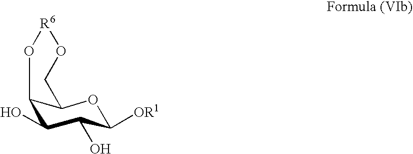

wherein R⁶ represents a ketal or acetal type protecting group selected from benzylidene, 4-nitrobenzylidene, 4-methoxybenzylidene or isopropylidene. In a preferred embodiment R⁷ represents a benzylidene protecting group.

The reaction is preferably carried out in a dipolar aprotic solvent such as dimethyl formamide (DMF) or acetone in the presence of acid catalysts such as p-toluene sulfonic acid or camphorsulfonic acid using a 2,2-dialkyloxypropane or an optionally substituted dialkyloxybenzylidene such as preferably benzaldehyde dimethyl acetal.

Suitable reaction temperatures range from ambient temperature to eievated temperatures. Preferably the reaction is carried out at a temperature of 25° C.

The compound of formula (VIb) may be then reacted with a compound of general formula (VIIb):

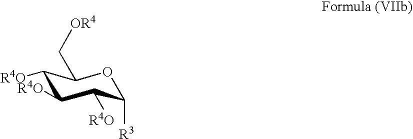

under substantially the same conditions as described above for the preparation of the compound of formula (IVa). In general formula (VIIb) R³ and R⁴ are as defined above. Selective glycosylation at the more reactive 3-position of the galactose may be achieved at reduced temperature such as 0° C. or lower, more preferably −10° C. or lower. Most preferably the reaction is carried out at about −20° C.

Resulting compound of general formula (VIIIb):

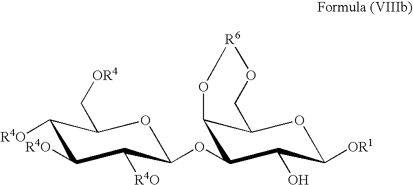

wherein R¹, R⁴ and R⁶ are as defined above, may subsequently be reacted with a compound of formula (VIIa) as defined above under substantially the same conditions as described above for the preparation of the compound of formula (IVa) to yield a compound of general formula (IXb):

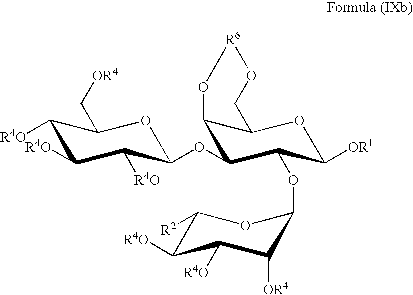

wherein R¹, R², R⁴ and R⁶ are as defined above.

Subsequently, the compound of formula (IXb) may be deprotected to yield the compound of formula (Ib). For example, the ester type protecting group R⁴ may be removed at pH 10-11 under substantially the same conditions as described above for the preparation of the compound of formula (Va). The reaction may then be neutralized by addition of solid carbon dioxide. On the other hand, R⁶ may be removed by using catalytic hydrogenation over palladium on carbon and hydrogen in an appropriate solvent such as ethanol or methanol. It should be understood that the removal of R⁴ and the removal of R⁶ are reversable.

1. A method for the preparation of a steroid modified chacotriose of general formula (Ia) or a steroid modified solatriose of general formula (Ib): 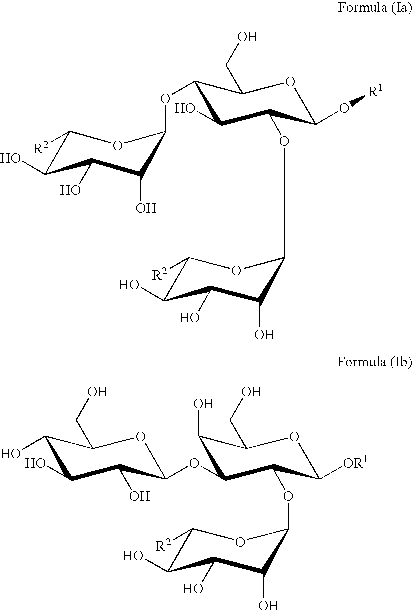
wherein R¹ represents a steroid or a derivative thereof having a hydroxyl group in the 3-position and no further unprotected hydroxyl groups; and each R² independently represents a straight or branched C₁₋₁₄ alkyl group, a C₅₋₁₂ aryl or heteroaryl group optionally substituted by one or more halogen atoms or C₁₋₄ alkyl groups, or a hydroxyl group, which method comprises the step of:
reacting a compound of general formula (IIa) or (IIb) 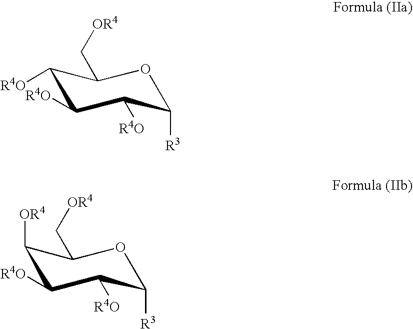
wherein R³ represents a halogen atom, an ethylsulfide or a sulfide group; and each R⁴ independently represents a benzoyl, substituted benzoyl, whereby the substituents are selected from C₁₋₄ alkyl groups, halogen atoms and NO₂, acetyl or pivolyl protecting group;
with a compound of general formula (III):
HO—R¹ Formula (III)
wherein R¹ is defined as above;
to yield a compound of general formula (IVa) or (IVb): 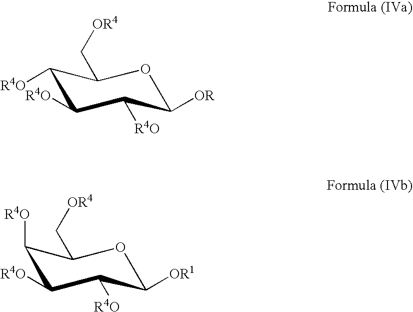
wherein R¹ and R⁴ are defined as above.

2. The method according to claim 1, further comprising the step of: deprotecting the compound of general formula (IVa) or (IVb), respectively, as defined in claim 1 to yield a compound of general formula (Va) or (Vb): 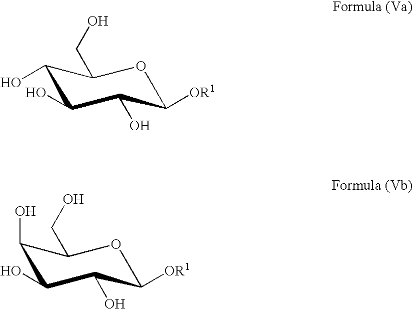
wherein R¹ is as defined in claim 1.

3. The method according to claim 2 for preparing a steroid modified chacotrose of general formula (Ia), further comprising the step of:
reacting the compound of general formula (Va) as defined in claim 2 with pivolyl chloride in the presence of an amine base to yield a compound of general formula (VIa): 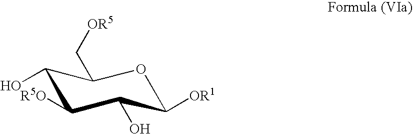
wherein R¹ represents a steroid or a derivative thereof having a hydroxyl group in the 3-position and no further unprotected hydroxyl groups, and R⁵ represents a pivolyl protecting group.

4. The method according to claim 3 for preparing a steroid modified chacotriose of general formula (Ia), further comprising the step of:
reacting the compound of general formula (VIa) as defined in claim 3 with a compound of general formula (VIIa): 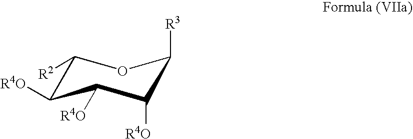
wherein each R² independently represents a straight or branched C₁₋₁₄ alkyl group, a C₅₋₁₂ aryl or heteroaryl group optionally substituted by one or more halogen atoms or C₁₋₄ alkyl groups, or a hydroxyl group;
R³ represents a halogen atom, an ethylsulfide or a sulfide group; and
each R⁴ independently represents a benzoyl, substituted benzoyl, whereby the substituents are selected from C₁₋₄ alkyl groups, halogen atoms and NO₂, acetyl or pivolyl protecting group;
to yield a compound general formula (VIIIa): 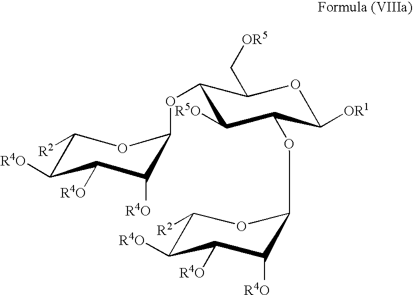
wherein R¹, R² and R⁴ are as defined above, and R⁵ is as defined in claim 3.

5. The method according of claim 4 for preparing a steroid modified chacotriose of general formula (Ia), further comprising the step of:
deprotecting the compound of general formula (VIIIa) as defined in claim 4 to yield the compound of general formula (Ia).

6. The method according to claim 2 for preparing a steroid modified solatriose of general formula (Ib), further comprising the step of:
selectively protecting the OH groups in and 4- and 6-position of the compound of formula (Vb) as defined in claim 2 with a ketal or acetal protecting type protecting group using standard conditions, to yield a compound of general formula (VIb): 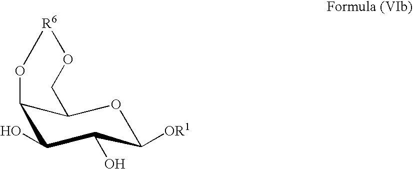
wherein R¹ represents a steroid or a derivative thereof having a hydroxyl group in the 3-position and no further unprotected hydroxyl groups; and R⁶ represents a ketal or acetal type protecting group selected from the group consisting of benzylidene, 4-nitrobenzylidene, 4-methoxybenzylidene and isopropylidene.

7. The method according to claim 6 preparing a steroid modified solatriose of general formula (Ib), further comprising the step of: reacting a compound of formula (VIb) as defined in claim 6 with a compound of general formula (VIIb): 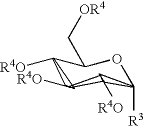
wherein R³ represents a halogen atom, an ethylsulfide or a sulfide group; and each R⁴ independently represents a benzoyl, substituted benzoyl, whereby the substituents are selected from C₁₋₄ alkyl groups, halogen atoms and NO₂, acetyl or pivolyl protecting group; to yield a compound of the general formula (VIIIb): 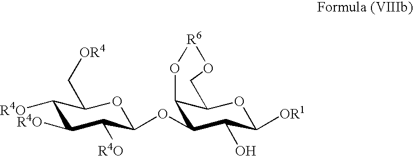
wherein R¹ represents a steroid or a derivative thereof having a hydroxyl group in the 3-position and no further unprotected hydroxyl groups; R⁴ is as defined
above; and R⁶ is as defined in claim 6.

8. The method according to claim 7 for preparing a steroid modified solatriose of general formula (Ib), further comprising the step of:
reacting a compound of formula (VIIIb) as defined in claim 7 with a compound of formula (VIIa) 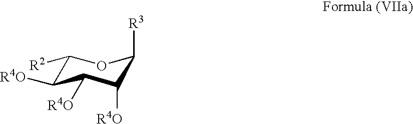
wherein each R² independently represents a straight or branched C₁₋₁₄ alkyl group, a C₅₋₁₂ aryl or heteroaryl group optionally substituted be one or more halogen atoms or C₁₋₄ alkyl groups, or a hydroxyl group;
R³ represents a halogen atom, an ethylsulfide or a sulfide group; and
each R⁴ independently represents a benzoyl, substituted benzoyl, whereby the substituents are selected from C₁₋₄ alkyl groups, halogen atoms and NO₂, acetyl or pivolyl protecting group; 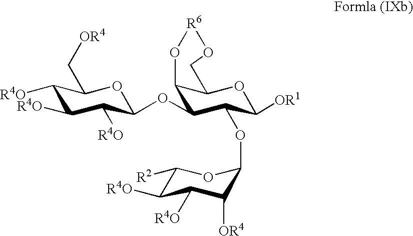
wherein R¹ represents a steroid or a derivative thereof having a hydroxyl group in the 3-position and no further unprotected hydroxyl groups; R² and R⁴ are as defined above; and R⁶ represents a ketal or acetal type protecting group selected from the group consisting of benzylidene, 4-nitrobenzylidene, 4-methoxybenzylidene and isopropylidene.

9. The method according to claim 8 for preparing a steroid modified solatriose of general formula (Ib), further comprising the step of:
deprotecting the compound of formula (IXb) as defined in claim 8 to yield the compound of formula (Ib).

10. The method according to claim 1, wherein R² represents a tomatidin-3-yl, demissidin-3-yl, solanidin-3-yl and solasodin-3-yl group.

11. The method according to claim 1, wherein R² represents a methyl group.

12. The method according to claim 1, wherein R³ in the compounds of formulae (IIa), and/or (IIb) represents a bromine atom.

13. The method according to claim 1, wherein the reaction is carried out in the presence of a promoter.

14. The method according to claim 13, wherein the promoter is selected from the group consisting of silver triflate, boron trifluoride diethyl etherate, trimethylsilyl triflate bromide, N-iodosuccinimide and dimethyl thiomethyl sulfonium triflate.

15. The method according to claim 14, wherein the promoter is silver triflate.

16. The method according to claim 1, wherein the reaction is carried out under anhydrous conditions in the presence of 4 Å mol sieves.

17. The method according to claim 2, wherein deprotection is carried out in dichloromethane or tetrahydrofuran in the presence of a C₁₋₄ alcohol and an alkali metal alkoxide having 1 to 4 carbon atoms.

18. The method according to claim 17, wherein deprotection is carried out in dichloromethane in the presence of methanol and sodium methoxide.

19. The method according to claim 2, wherein deprotection is carried out in dichloromethane or tetrahydrofuran in the presence of water, an alkali metal hydroxide and a C₁₋₄ alcohol.

20. The method according to claim 19, wherein deprotection is carried out in tetrahydrofuran, and wherein the alkali metal hyderoxide is sodium hydroxide and the alcohol is methanol.

21. The method according to claim 1 for preparing a steroid modified solatriose of general formula (Ib), wherein R⁴ represents a benzoyl or p-toluolyl protecting group.

22. The method according to claim 1, wherein reacting a compound of general formula (IIa) or (IIb) with a compound of general formula (III) is carried out in the presence of sterically hindered non-nucleophilic base.

23. The method according to claim 22, wherein the sterically hindered non-nucleophilic base is selected from 2,6-lutidine, 2,4,6-collidine or 2,6-di-tertbutyl-4-methyl pyridine.

24. A steroid modified chacotriose of general formula (Ia) as defined in claim 1, wherein R¹ represents a tomatidin-3-yl or demissidin-3-yl group.

25. A compound of general formula (VIIIa) as defined in claim 4.

26. A compound of general formula (VIIIb) as defined in claim 7.

27. A compound of general formula (VIa) as defined in claim 3.

28. A compound of general formula (VIb) as defined in claim 6.

29. A compound of general formula (Va) or (Vb) as defined in claim 2.

30. A compound of general formula (IVa) or (IVb) as defined in claim 1.

31. A compound of general formula (IXb) as defined in claim 8.
